(19) United States
(12) Patent Application Publication
(10) Pub. No.: US 2007/0275388 A1
(43) Pub. Date: Nov. 29, 2007

(54) Dendrimers that possess a single target annealing site and uses thereof

(76) Inventor: Daniel Ryan, San Francisco, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/440,920

(22) Filed: May 25, 2006

(51) Int. Cl. C12Q 1/68; C07H 21/04

(52) U.S. Cl. 435/6; 536/25.32; 977/924

(57) ABSTRACT

Provided is a polynucleotide dendrimer having a single unique binding site for binding a target molecule, thereby permitting stoichiometric quantification of the dendrimer-bound target molecule. The dendrimer of the invention is useful in conjunction with various methods for detecting, and optionally quantifying, nucleic acids, proteins, polysaccharides, organic compounds, or antigens, among others.

### BACKGROUND OF THE INVENTION

Labeled polynucleotide dendrimers for detection of nucleic acids have been described in the literature and are commercially available, for instance, from Genisphere® (Hatfield, Pa.). Polynucleotide dendrimers are typically composed of annealed and covalently cross-linked strands of DNA. See, for instance, Nilsen et al., J. Theor. Biol. 187:273-284 (1997) and U.S. Pat. Nos. 5,175,270 and 6,274,723.

Such polynucleotide dendrimers are typically prepared by protocols having the following features. (i) The starting material is a double-stranded duplex of DNA with 5′ and 3′ single-stranded overhangs, or “binding arms,” attached to the duplex (e.g., four binding arms total), called the “initiating monomer,” which is descriptive of its role in the assembly of a dendrimer. Each initiating monomer's 5′ and 3′ binding arms are annealed to complementary binding arms on “extending monomers” that have similar composition and morphology. (ii) A subset of the four binding arms on each extending monomer is complementary to the binding arms on the initiating monomer. The non-complementary binding arms of the extending monomers are inactive for annealing to the initiating monomer. Typically, four extending monomers can anneal to the initiating monomer to yield a single-layer, or one-layer, dendrimer in solution. (iii) To add another layer of extending monomers to dendrimers, one typically adds similar but distinguishable extending monomers, in which each monomer has a subset of its four binding arms that is complementary to binding arms on the dendrimer. Thus, a one-layer dendrimer can be converted to a two-layer dendrimer, and so on, stepwise, until a desired size of dendrimer is reached. Typically, dendrimers of three or four layers are used for the detection of nucleic acids. (iv) For detection of nucleic acids, the outer layer of the dendrimers in prior art has at least two types of binding arms: (1) one type of binding arm anneals to an oligonucleotide that is attached to a detectable chemical moiety, or “label,” such as a photon-emitting molecule, e.g. a fluorophore, and (2) another type of binding arm, a “target-annealing sequence,” is designed for specific annealing to a target sequence of interest. The photon-emitting dendrimers are added to a sample that contains nucleic acids, and the target-annealing sequence of the dendrimers is annealed to the target sequence in the sample. Unbound dendrimers are separated from the target-bound dendrimers, typically by prior or subsequent binding of the target or targets to a washable solid support that withstands the washing conditions for removing unbound dendrimers. After the washing, the prior art allows one to detect the target-bound dendrimers via spectroscopic methods.

Photon-emitting dendrimers facilitate spectroscopic detection of the dendrimer-bound nucleic acid targets, in part by providing a target-specific spectroscopic signal whose spectral properties are characteristic of the dendrimer and distinct from those of nucleic acids and other molecules in the spectroscopic sample. The spectral properties of the dendrimers enable detection of low abundance targets. See, for instance, Stears et al., Physiol. Genomics 3:93-99 (2000). Polynucleotide dendrimer technology has been found to be useful for rapid and accurate pathogen identification (Borucki et al., J. Clin. Microbiol. 43:3255-3259 (2005), diagnostic applications (Capaldi et al., Nuc. Acids Res. 28:e21 (2000)) and for formalin-fixed, paraffin-embedded (FFPE) RNA detection. Dendrimers have been used with suspension arrays (Borucki et al., J. Clin. Microbiol. 43:3255-3259 (2005). Polynucleotide dendrimers have also been used to detect proteins (Kadushin et al., “Enhancement of Sensitivity in Luminex Protein Detection Assays via Dendrimer Dependent Signal Amplification,” Poster [online], Luminex Planet xMAP Conference, Austin, Tex. Apr. 25-27, 2005 [retrieved 2006-4-11]. Retrieved from the Internet: www(dot)genisphere(dot)com/pdf/Genisphere_Luminex_Planet_xMAP_{—}042505(dot )pdf).

A major disadvantage of the target-binding dendrimers in prior art is that the combinatorial methods for preparing them are designed to furnish the outer layer of the dendrimers with multiple copies of the target-annealing sequence. Therefore, the dendrimers can bind multiple copies of a target, which dilutes the photoemission intensity, or signal intensity, per copy of bound target. The more targets bound to a single dendrimer, the greater the dilution of signal intensity per copy of bound target. Furthermore, quantification of the target by the prior art is not practical. Although one can measure the signal intensity of bound dendrimer, this measurement does not allow one to determine how many target molecules are bound to the dendrimer.

In light of these findings, it is clear that, prior to the present invention, there was an unmet need in the art for a polynucleotide dendrimer that permits quantification of a target molecule, rather than merely detection of the target molecule. The instant invention meets this need.

### SUMMARY OF THE INVENTION

The present invention provides a polynucleotide dendrimer having only a single target-annealing site, comprising a polynucleotidal initiating monomer having a single-stranded target-annealing site on or protruding beyond the exterior layer of the dendrimer and a plurality of polynucleotidal extending monomers, wherein the initiating monomer and the plurality of extending monomers are joined by hybridization. In one embodiment, each extending monomer comprises initially a double-stranded trunk flanked by at least two hybridization binding arms that are single-stranded, such that when incorporated into the polynucleotide dendrimer, at least one of the hybridization binding arms of each extending monomer hybridizes to a complementary binding arm of the initiating monomer or another extending monomer. In addition, the dendrimers may be labeled, such as by: chromophore, chromogen, fluorophore, phosphor, radioactive moiety, biotin, antigen epitope, polynucleotidal aptamer, isotope detectable by mass spectrometry, or isotope detectable by nuclear magnetic resonance spectroscopy. Further, the dendrimer may comprise intermolecular crosslinks.

The invention further provides a polynucleotidal initiating monomer useful in preparing a dendrimer, comprising a trunk, at least one single-stranded hybridization binding arm covalently attached to the trunk and a polynucleotidal extension arm covalently attached to the trunk, wherein the extension arm comprises a double-stranded extender portion and a single-stranded target-annealing site.

The invention further provides a method of making a polynucleotide dendrimer, the method comprising a polynucleotidal initiating monomer of the invention and a plurality of extending monomers, wherein the extension arm comprises a double-stranded extender portion and a single-stranded target-annealing site and wherein each extending monomer comprises initially a double-stranded trunk flanked by at least two hybridization binding arms that are single-stranded, such that when incorporated into the polynucleotide dendrimer, at least one of the hybridization binding arms of each extending monomer hybridizes to a complementary binding arm of the initiating monomer or another extending monomer.

The invention also provides a method of detecting a target molecule comprising the steps of contacting a dendrimer of the invention with a sample containing a target molecule, binding the dendrimer to the target molecule and detecting the dendrimer, thereby detecting the bound target molecule.

In addition, the invention provides kits containing a dendrimer of the invention. In one embodiment, the kit further includes an oligomer comprising a sequence complementary to a hybridization binding arm of the extending monomer of the exterior layer of the dendrimer, and a detectable label. Optionally, the kit further includes a target probe, wherein the target probe comprises a sequence that is complementary to the target-annealing site and a target-detecting portion.

In one embodiment, the target-detecting portion is a nucleic acid sequence that is complementary to a portion of a primer and wherein the kit further comprises the primer.

The invention also provides a composition useful for detecting two different target molecules in a sample simultaneously, the composition comprising a first polynucleotide dendrimer and a second polynucleotide dendrimer, wherein: each polynucleotide dendrimer comprises a detectable label and a polynucleotidal initiating monomer having a single-stranded target-annealing site projecting beyond the dendrimer's exterior layer and a plurality of polynucleotidal extending monomers; the initiating monomer and the plurality of extending monomers are joined by hybridization; the single-stranded target-annealing site of the first polynucleotide dendrimer is different from the single-stranded target-annealing site of the second polynucleotide dendrimer; and the detectable label of the first polynucleotide dendrimer is distinguishable from the detectable label of the second polynucleotide dendrimer.

Additional objects, advantages and novel features of the invention will be set forth in part in the description, examples and figures which follow, all of which are intended to be for illustrative purposes only and not intended in any way to limit the invention, and in part will become apparent to those skilled in the art on examination of the following, or may be learned by practice of the invention.

### BRIEF DESCRIPTION OF THE DRAWINGS

FIGS. 1A-1C schematically depict a polynucleotidal extension arm (1A) and a target probe (1B), and an initiating monomer comprising three binding arms (1C). Cross-hatching indicates hybridization between strands.

FIGS. 2A to 2F schematically depict various initiating monomers of the invention having a double-stranded polynucleotide core and one or two binding arms. FIGS. 2A-2C depict three initiating monomer variants comprising one binding arm. FIGS. 2D-2F depict three initiating monomer variants comprising two binding arms. Cross-hatching indicates hybridization between strands.

FIG. 3 is a schematic of an extending monomer. Cross-hatching indicates hybridized sections between strands.

FIG. 4 is a schematic of a dendrimer of the invention having one layer of extending monomers hybridized to an initiating monomer of the invention. Note that the scale of the polynucleotidal extension arm is compressed with respect to the other nucleic acid structures depicted. Cross-hatching indicates hybridization between strands. Hybridization between binding arms of different monomers is indicated by narrower cross-hatching.

FIG. 5 is a schematic of a portion of a dendrimer of the invention having an inner layer of extending monomers, 34 and a portion of an outer layer of different extending monomers, 34′. Binding arms on the outer layer, comprising monomers 34′, are hybridized to discrete oligomers, each of which is attached to a detectable label, represented by a black circle. Cross-hatching indicates hybridization between strands. Hybridization between binding arms of different monomers, and between binding arms and the oligomer bearing a detectable label, is indicated by narrower cross-hatching.

### DETAILED DESCRIPTION OF CERTAIN EMBODIMENTS

The invention is a polynucleotide dendrimer, a type of polynucleotide matrix, that has on or protruding from beyond its exterior surface only a single-stranded binding site that is unique in and characteristic of the dendrimer. The invention is further drawn to a novel initiating monomer comprising the single-stranded binding site, as the novel monomer is both essential and useful for making the dendrimer of the invention. The invention is further drawn to a method of using the dendrimer to detect a molecule of interest and a kit comprising the polynucleotide dendrimer.

Definitions for terms used in the present application are now provided, followed by the Detailed Description of the invention.

### Definitions

As used herein, a “polynucleotide dendrimer” or “dendrimer” refers to a matrix of polynucleotides, exhibiting regular branching, formed by the sequential or generational addition of branched layers to or from a core molecule, such as an initiating monomer.

As used herein, an “initiating monomer” is a polynucleotidal compound that serves to nucleate the formation of a dendrimer.

As used herein, an “extending monomer” is a polynucleotidal compound that can bind to the initiating monomer and/or to each other during assembly of a dendrimer. Extending monomers form the layers of the dendrimer. The first layer of a dendrimer is the layer of extending monomers closest to the initiating monomer. The outer layer is the layer furthest from the initiating monomer and forming the surface of the dendrimer. Extending monomers are also referred to in the art as matrix monomers, matrix extending monomers and matrix polynucleotide monomers.

As used herein, a “trunk” is the double-stranded portion of an extending monomer that is flanked by two or more hybridization binding arms. “Trunk” also refers to the double-stranded portion of an initiating monomer which is flanked by one or more hybridization binding arms. A trunk is double stranded whether the monomer is in a dendrimer or not. The double-stranded trunk is formed when the strands of a monomer are hybridized together.

A “hybridization binding arm” (also referred to as a “binding arm”) refers herein to a sequence of nucleotides in an initiating or extending monomer that is capable of hybridizing to a complementary binding arm of a different monomer during assembly of the monomers into a dendrimer. In the extending monomers of the last layer of a dendrimer, some binding arms are intended to be labeled or hybridized to oligonucleotides that each possess a detectable label, thereby labeling the dendrimer.

The term “sample” as used herein relates to a material or mixture of materials, containing one or more components of interest. Samples include, but are not limited to, samples obtained from a chemical or biochemical reaction or series of reactions, from an organism or from the environment (e.g., a soil sample, water sample, etc.) and can be directly obtained from a source (e.g., such as a biopsy or from a tumor) or indirectly obtained, e.g., after culturing and/or one or more processing steps. In one embodiment, samples are a complex mixture comprising, e.g., about 10 or more different molecules, about 60 or more different molecules, about 200 or more different molecules, about 500 or more different molecules, about 1000 or more different molecules, about 4000 or more different molecules, about 10,000 or more molecules, etc.

The term “conditions” refers to the chemical and physical properties of various embodiments and/or of their immediate surroundings, comprising, but not limited to, the solvent(s) employed, including any dissolved salts, buffers, reagents, dissolved gases and/or non-solvent molecules, temperature, pressure, any reactive chemicals in solution or in suspension or at the interface of the solution and an insoluble phase.

As used herein, “attached” or “bound” refers to the effects of a chemical bond, or group of bonds, that resists disruption under the conditions the bond or group of bonds is subjected to in the course of standard use, or in a defined period, or under defined conditions. Such a bond or group of bonds can comprise covalent and/or non-covalent bonds, including hydrogen bonds. Molecules bound under defined conditions may cease to be bound if the conditions are substantially changed, e.g., by raising the temperature or altering the salt concentration of a solution of bound molecules.

A “biomonomer” references a single molecular unit that can be covalently linked with the same or other biomonomers to form a biopolymer. A biomonomer can be produced from controlled decomposition of a biopolymer. An example of a biomonomer is a single amino acid or a nucleotide, each of which has two chemical moieties that can be activated to form covalent bonds between the same or similar monomers. One or both of the chemical moieties may include removable protecting groups.

A “biopolymer” is a biological polymer of one or more types of repeating units, or monomers. Biopolymers are typically found in biological and biochemical systems and particularly include polysaccharides (such as carbohydrates), peptides (which term is used to include polypeptides and proteins, whether or not attached to a polysaccharide) and polynucleotides, as well as their analogs such as those compounds composed of or containing amino acid analogs or non-amino acid groups, or nucleotide analogs or non-nucleotide groups. The term “biopolymer” includes various polynucleotides: deoxyribonucleic acid or DNA (including cDNA), ribonucleic acid or RNA (including mRNA, cRNA, snRNA, hnRNA, siRNA, and miRNA), and oligonucleotides, regardless of their source.

A “nucleotide” refers to a subunit of a polynucleotide, or nucleic acid, and has a ribose sugar ring, a basic pyrimidine or purine ring, and a phosphate group comprising the 5′-hydroxyl oxygen of the ribose ring. As used herein, “nucleotide” also refers to functional and/or structural analogs of such subunits, whether the analogs are synthetic or natural, which in polymeric form can hybridize to other polynucleotides in a sequence-specific manner analogous to that of Watson-Crick-type base pairing. Nucleotide subunits of DNA are deoxyribonucleotides, and nucleotide subunits of RNA are ribonucleotides. The five most common nucleotides are adenine, guanine, thymidine, cytidine and uracil.

An “oligonucleotide” generally refers to a nucleotide oligomer that is relatively short, composed of about 2 to about 200 nucleotides, e.g., about 10 to about 100 nucleotides, or about 30 to about 80 nucleotides. Oligonucleotides may be synthetic. Oligonucleotides may comprise a nucleotide analog or analogs, e.g., PNA, LNA and/or UNA molecules.

A “polynucleotide” or “nucleic acid” refers to a nucleotide multimer of any number of nucleotides. These terms includes structural analogs in which some or all of the component biomonomers are non-natural or synthetic, in compliance with the criterion that at least some of the biomonomers are capable of participating in Watson-Crick-type hydrogen bonding interactions, or similar hydrogen bonding interactions. Polynucleotides can exist in single- or multi-stranded conformations, and where one or more of the strands may or may not be completely aligned with another of the strands.

As used herein, “primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. A “random primer” as used herein refers to one of a mixture of polynucleotides of a given number of nucleotides representing all possible sequences for that number of nucleotides.

The terms “ribonucleic acid,” “polyribonucleotide” and “RNA” as used herein mean a biopolymer, whether naturally occurring or synthetic, composed of ribonucleotides.

The terms “deoxyribonucleic acid” and “DNA” as used herein mean a biopolymer, whether naturally occurring or synthetic, composed of deoxyribonucleotides.

The term “mRNA” means messenger RNA.

The terms “complementary” and “complementarity” refer to the broad concept of a property of polynucleotides that allows and/or promotes base-specific hydrogen bonding (namely, “base pairing”) between two nucleotides or between several nucleotides in two distinct regions. It is known that an adenine nucleotide in the first region is capable of forming specific hydrogen bonds (i.e., base pairs) with a nucleotide in the second region if the nucleotide is thymidine or uracil and if the thymidine and adenine interaction, or the uracil and adenine interaction, can conform to an anti-parallel geometry that allows base pairing. Similarly, it is known that a cytidine in the first region is capable of base pairing with a nucleotide in the second region, which is anti-parallel to the first region, if the nucleotide is guanine. The first polynucleotide region is complementary to the second region if, when the two regions are arranged in an anti-parallel fashion, at least one nucleotide in the first region is capable of base pairing with a particular type of nucleotide in the second region. Preferably, when the first and second regions are arranged in an anti-parallel fashion, at least about 50%, and more preferably at least about 75% or about 100%, of the nucleotides in the first region are capable of base pairing with nucleotides in the second region. In some embodiments, all nucleotides of the first region are capable of base pairing with nucleotides in the second region.

As used herein, “hybridization,” “hybridize,” “annealing” and “anneal” refer to the formation of hydrogen bonds during Watson-Crick-type base pairing of complementary oligonucleotide sequences oriented in anti-parallel fashion.

As used herein, “antibody” refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. Antibodies useful in the present invention can exist in a variety of forms including, for example, polyclonal antibodies, monoclonal antibodies, Fv, Fab and F(ab)₂, as well as single chain antibodies (scFv) and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., Proc. Natl. Acad. Sci. USA 85:5879-5883 (1988); Bird et al., Science 242:423-426 (1988)).

An “antigen” can be any molecule that is immunogenic. Non-limiting examples of antigens include: proteins, nucleic acid, steroids, prostaglandins, cytokines, cell surface proteins such as immunoglobulins and cell adhesion molecules, lipids, fatty acids, tumor markers, cell-type specific markers, pathogens, antigenic components of pathogens, toxins, amino acids, cellular-compartment specific markers, carbohydrates, and polysaccharides, among others.

As used herein, “aptamer” refers to small molecule that can bind specifically to another molecule. Aptamers are typically either polynucleotide or peptide. A polynucleotidal aptamer are DNA or RNA molecules, usually comprises several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold, that bind to specific ligands. Peptide aptamers may be identified and isolated from combinatorial libraries, using methods such as the yeast two-hybrid system.

A molecular “array” or “microarray,” unless a contrary intention appears, includes any one, two or three-dimensional arrangement of addressable regions, each of which bears a particular molecule or molecules (e.g., biopolymers comprising polynucleotide sequences), where the molecules are immobilized on the surface of a solid support. By “immobilized” is meant that the molecules are stably associated with the surface, such that they do not separate from the surface under conditions of using the array, e.g., under hybridization, washing and/or stripping conditions. As is known in the art, the moiety or moieties may be covalently or non-covalently bound to the surface in the addressable regions. An array may contain more than ten or even more than one million discrete addressable regions, or “features,” in an area of less than 20 cm². Each feature may have a width (i.e., diameter, for a round region) in the range of from about 1 μm to about 1.0 cm. In general, an array feature is nearly homogeneous in composition. A given feature is composed of identical (or nearly identical) molecules, e.g., nucleic acids of identical sequence, that can bind to (e.g., hybridize to) a distinct molecule or molecules (i.e., the target) which may be dissolved in the solution used to wet the array (i.e., the array is immersed in the solution). If the target molecule is present in the solution, then its respective feature can bind to the target molecule and sequester it from the solution. In most arrays, the features of an array are approximately homogenous in concentration on the solid support. Inter-feature areas are typically (but not essentially) present in arrays, and these areas do not bind targets, at least not nearly to the extent that features can. It will generally be appreciated that the inter-feature areas, when present, could be of various sizes and shapes. An array is “addressable” in that the location and target-binding specificity of each feature is pre-determined and charted, before the array is produced. Therefore, the target for which each feature is specific is known to the practitioner. The term “array” encompasses bead arrays.

As used herein, a “polynucleotide within a polynucleotide array” refers to either a polynucleotide that is part of an array or to a polynucleotide that is hybridized to a polynucleotide that is part of an array.

The phrase “solid support” refers to a solid and/or to the surface of the solid which does not dissolve, melt, sublime, crack, disintegrate or cause undesired chemical reactions under the conditions of its use, which typically involve the formation of molecular complexes and/or chemical bonds on or near the surface of the solid. In all cases, molecules that are distinct from the solid itself are deposited onto and/or bind to the surface the solid, whether prior to its intended use or during its intended use or both. The solid can have a variety of configurations, e.g., a sheet, bead, particle, slide, wafer, web, fiber, tube, capillary, microfluidic channel or reservoir, or other structure. The support can be planar, non-planar or a combination thereof. The support can be porous or non-porous. In certain embodiments, oligonucleotides are deposited on the surface of the solid, e.g., in the form of an array. Glass is the most common solid support for arrays, although fused silica, silicon, plastic and other materials are also suitable.

“Addressable” regions or features, and analogous terms, refer to the known locations of different molecular moieties of known characteristics (e.g., nucleotide sequence composition) that are attached or adhered to, or that are intended to be attached or adhered to, the surface of a solid support, such that each known location is associated with a known molecular moiety that possesses a characteristic binding affinity, and such that one can identify a molecular target based on the location(s) on the solid surface where the target binds under stringent conditions.

In certain embodiments, an array is coated with or otherwise contacted with a sample that comprises one, several or very many different biopolymers under “stringent conditions,” i.e., conditions that allow non-covalent binding between two biopolymers of sufficient mutual affinity to yield the desired level of binding specificity in the assay, while inhibiting or precluding binding between biopolymers of insufficient mutual affinity. Stringent assay conditions comprise binding conditions and wash conditions for removing unbound molecules from the location(s) of binding.

“Stringent” assay conditions and “stringency” refer to conditions at the time of the assay and/or in preparation for the assay that restrict the formation of complexes that lack sufficient affinity to comply with the desired specificity for complex formation. Under conditions referred to as stringent, the formation of so-called non-specific complexes is typically less than about 50% of the smallest quantity of any specific complex that is or that can be detected by the assay, according to the desired specificity, e.g., conditions that produce quantities of non-specific complexes that are undetectable or may be detectable at less than asignal-to-noise ratio of about 2 when the detection method for the assay is used. Other stringent binding conditions are known in the art and may also be employed, as appropriate.

Stringent conditions may also include a “pre-binding” or “pre-hybridization” step in which an agent that binds to molecular regions that possess binding affinities that are not of interest or that interfere with the assay is added to an aqueous solution of the sample and/or to a liquid interface that wets features bound to a solid support, and in which the addition occurs prior to joining the aqueous sample with the solid support-bound features. For example, certain stringent hybridization conditions include, prior to any hybridization of the sample's polynucleotides with the polynucleotides bound to a solid support, a step for hybridization of the sample with Cot-1 DNA, or the like.

As known in the art, “stringent hybridization conditions,” “stringency of hybridization,” “stringent wash conditions,” “stringency of washing,” “stringent assay conditions” and “stringency of the assay” in the context of nucleic acid hybridizations are sequence dependent, and are different under different experimental parameters. The terms refer to conditions that are compatible to produce complexes between complementary binding members, e.g., between immobilized features and complementary sample polynucleotides, but which does not result in any substantial formation of complexes between non-complementary polynucleotides (e.g., any formation of the latter complexes is not detected when array signals are normalized against background signals in inter-feature areas and/or in control regions on the array). Stringent hybridization conditions include, but are not limited to, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include hybridization in a buffer of 40% formamide, 1 M NaCl, and 1% SDS at 37° C., and a wash in 1×SSC at 45° C. Alternatively, hybridization in 0.5 M NaHPO₄, 7% sodium dodecyl sulfate (SDS), 1 mM EDTA at 65° C., and washing in 0.1×SSC, 0.1% SDS at 68° C. can be performed. Additional stringent hybridization conditions include hybridization at 60° C. or higher and 3×SSC (450 mM sodium chloride, 45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

Stringent wash conditions used to remove unbound polynucleotides may include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the bound polynucleotide complexes are washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC, 0.1% SDS at 42° C.

A specific example of stringent assay conditions is hybridization of rotating arrays at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature. Other methods of agitation can be used, e.g., shaking, spinning, and the like.

Additional hybridization methods are described in references describing array techniques (Kallioniemi et al., Science 1992;258:818-821 and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol. 1981;21:470-480 and Angerer et al., In Genetic Engineering: Principles and Methods, Setlow and Hollaender, Eds. Vol 7, pgs 43-65 (Plenum Press, New York 1985). See also U.S. Pat. Nos. 6,335,167; 6,197,501; 5,830,645; and 5,665,549; the disclosures of which are herein incorporated by reference.

The terms “determining,” “measuring,” “evaluating,” “assessing” and “assaying” and related terms are used interchangeably herein to refer to any form of measurement, and comprise determining if a molecule, molecular moiety or other distinct entity is present or not, and/or how much of the entity is present. These terms include both quantitative and/or qualitative determinations. Assessing may be relative or absolute. For example, “assessing the presence of” may include determining the amount of something present, as well as determining whether it is present or absent.

### DETAILED DESCRIPTION

The polynucleotide dendrimer of the invention has a defined binding stoichiometry. The defined binding stoichiometry results from assembling the dendrimer using a novel initiating monomer and a plurality of extending monomers, added sequentially or generationally. The novel initiating monomer has one single-stranded target-annealing site, which is designed to be at or protrude beyond the exterior surface of the dendrimer, and having a sequence that is unique in the dendrimer. Therefore, each dendrimer of the invention has a single, unique target-annealing site at or protruding beyond its surface. This single target-annealing site establishes the binding stoichiometry of the dendrimer as 1:1 (i.e., 1 target binding site per dendrimer). This defined stoichiometry is advantageous in at least two ways. Prior art polynucleotide dendrimers comprise multiple copies of the target-annealing sequence on each dendrimer. Because a prior art dendrimer can bind multiple copies of a target, the signal intensity per bound target can and does vary substantially. For instance, a single dendrimer may bind a few target molecules or it may bind many target molecules. In either case, however, the signal intensity from the single dendrimer is the same. By possessing a 1:1 stoichiometry for target binding, the polynucleotide dendrimers of the invention essentially increase the signal intensity per bound target, which thus facilitates detection of the bound target. Furthermore, the 1:1 stoichiometry enables the quantification of the bound target with greater accuracy and precision by substantially reducing and constraining a source of variability. Quantification using the dendrimers of the invention is limited only by the standard deviation in the calibration curves produced experimentally for measuring the signal intensity in, for example, spectrophotometric detection measurements.

The structural design and assembly of polynucleotide dendrimers is generally known in the art. See, for instance, U.S. Pat. Nos. 5,175,270 and 6,274,723, incorporated herein by reference in their entireties. The assembly of a dendrimer results in a three-dimensional shape, typically but not exclusively, a roughly spherical shape comprising layers of extending monomers. The initiating monomer constitutes the approximate center of a dendrimer, depending on the type of branching in the dendrimer. The three-dimensional assembly of extending monomers around the initiating monomer forms the interior volume of the dendrimer. The last, outer layer of extending monomers forms the surface of the dendrimer. In prior art polynucleotide dendrimers, however, the outer layer comprises numerous binding sites for a target molecule or molecules, as well as numerous sites for attachment of a detectable label. As discussed above, this is a disadvantage when the dendrimer is used in an application for quantifying the target molecule.

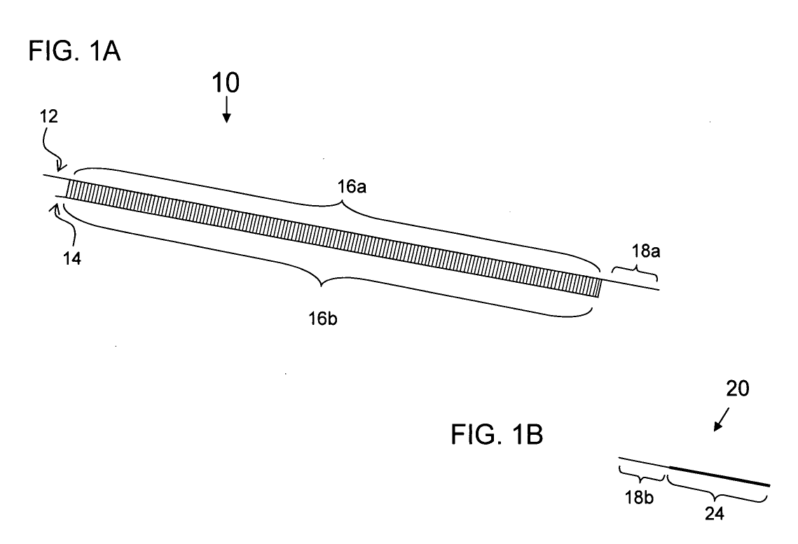

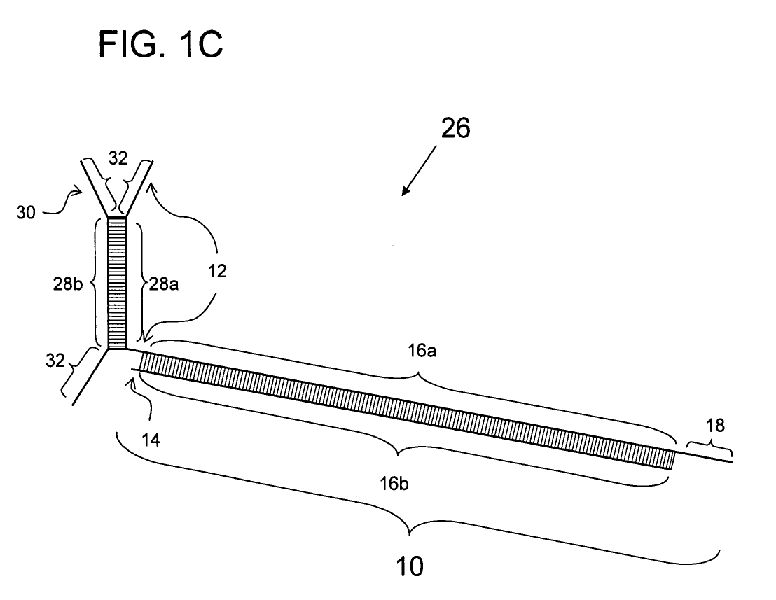

To provide the 1:1 binding stoichiometry, the polynucleotide dendrimer of the present invention comprises a unique initiating monomer having only one polynucleotidal extension arm, shown schematically in FIG. 1A. Polynucleotidal extension arm 10 is a portion of the initiating monomer comprising two polynucleotide strands, 12 and 14. Strand 12 continues into another portion of the initiating monomer, as depicted in FIG. 1C. In extension arm 10, strands 12 and 14 are extensively hybridized to each other. Strand 12 comprises extender portion 16a and target-annealing site 18. Strand 14 comprises extender complement 16b, that is complementary to extender portion 16a of strand 12. In the initiating monomer of the invention, extender complement 16b is hybridized to extender portion 16a to form double-stranded extender portion 16. Target-annealing site 18a is a polynucleotide sequence that binds specifically to a complementary sequence in a target molecule of interest. In one embodiment, target-annealing site 18a binds specifically to complementary sequence 18b in target-detecting probe 20, shown in FIG. 1B. Target-annealing site 18a ranges from about 15 to about 70 nucleotides, and preferably from about 20 to about 60 nucleotides in length.

Double-stranded extender portion 16 of extension arm 10 serves to position target-annealing site 18a of extension arm 10 at or protruding beyond the dendrimer surface, thereby enabling target-annealing site 18a to bind to a complementary sequence exterior to the polynucleotide dendrimer. Accordingly, double-stranded extender portion 16 of polynucleotidal extension arm 10 comprises a sufficient number of base pairs, such that target-annealing site 18a will be entirely exposed at, and preferably protrudes beyond the surface of the dendrimer.

Commercially-available dendrimers typically have 3 to 4 layers of extending monomers, and the approximately spherical dendrimers have an average radius of about 75 to about 100 nanometers. For target-annealing site 18a of polynucleotidal extension arm 10 to be at or protruding beyond the surface of a dendrimer of a similar radius, double-stranded extender portion 16 should be at least about 110 nm long. In B-DNA, which is entirely double-stranded and conforms to the most common naturally-occurring, or “B,” helical conformation, the DNA double helix has about 0.34 nm of axial length per base pair (i.e., distance on the linear axis of the DNA helix equal to a parallel distance that just spans the molecular dimensions of a mean-averaged base pair). Therefore, for a similarly structured double-stranded polynucleotide to have about 110 nm of axial length, it has about 325 contiguous base pairs. In an embodiment wherein the dendrimer has a radius of about 100 nm, double-stranded extender portion 16 of polynucleotidal extension arm 10 is at least about 325 base pairs long, preferably at least about 365 base pairs long, and more preferably, at least about 400 base pairs long.

In addition to the polynucleotidal extension arm, the initiating monomer of the invention comprises a double-stranded trunk portion, and at least one single-stranded hybridization binding arm. The single-stranded binding arm in an initiating monomer is intended to hybridize to a complementary single-stranded binding arm in an extending monomer during preparation of the polynucleotide dendrimer.

One embodiment of an initiating monomer of the invention is shown in FIG. 1C, wherein the trunk comprises portions 28a and 28b, which are hybridized to each other. In this embodiment, initiating monomer 26 is assembled from three polynucleotide strands, strands 12, 14 and 30. In general, strand 12 comprises extender portion 16a, flanked by target-annealing site 18a of extension arm 10 and trunk portion 28a, which constitutes half of double-stranded trunk 28. In the embodiment depicted in FIG. 1C, trunk portion 28a is further flanked by binding arm 32. Strand 30 of initiating monomer 26 comprises the other trunk portion 28b (which is complementary to trunk portion 28a), flanked by two binding arms 32. Note that although all binding arms are labeled with the same identifier, 32, this is not intended to indicate or imply that they have identical sequences. The common identifier merely indicates they have a common function. As previously described, strand 14 comprises extender complement 16b that is complementary to extender portion 16a of strand 12.

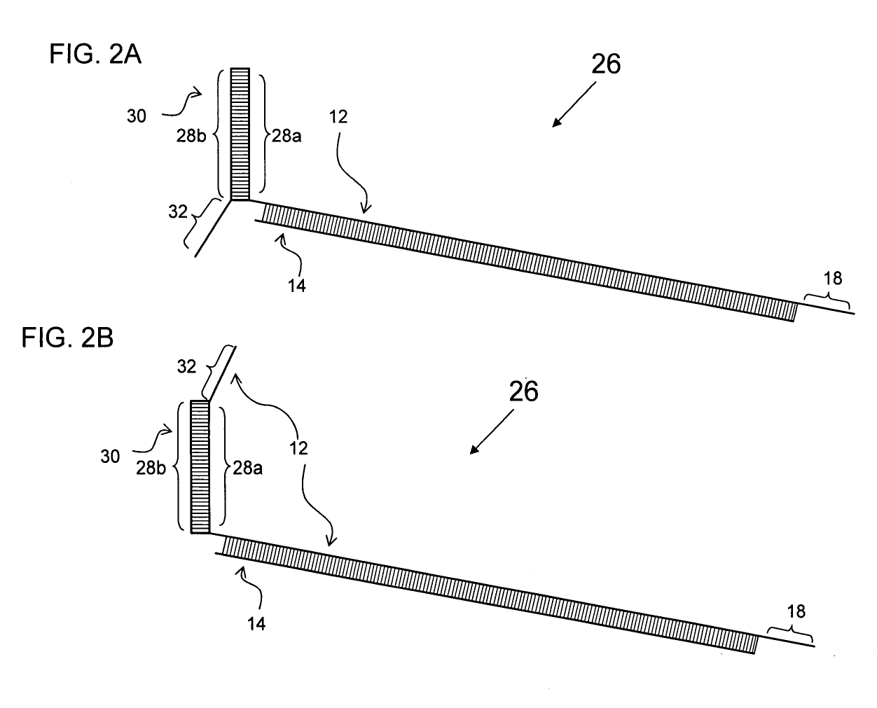

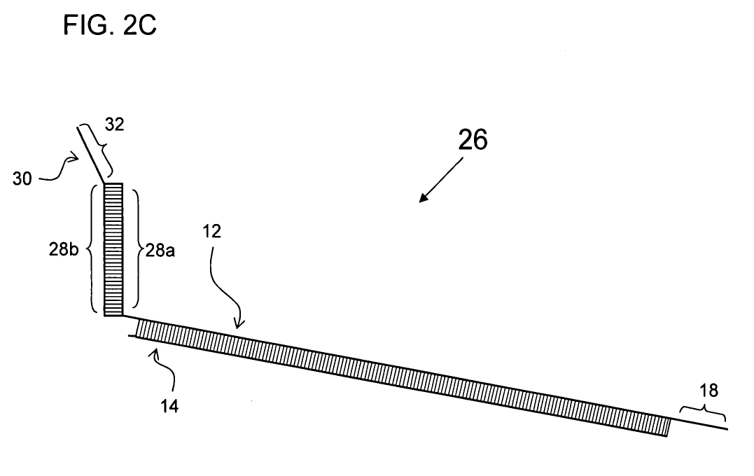

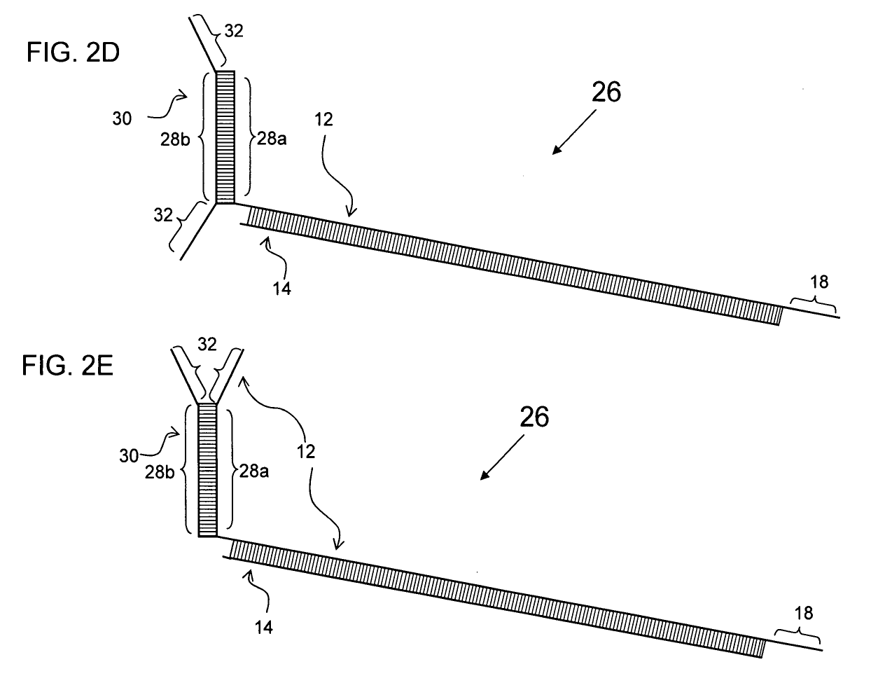

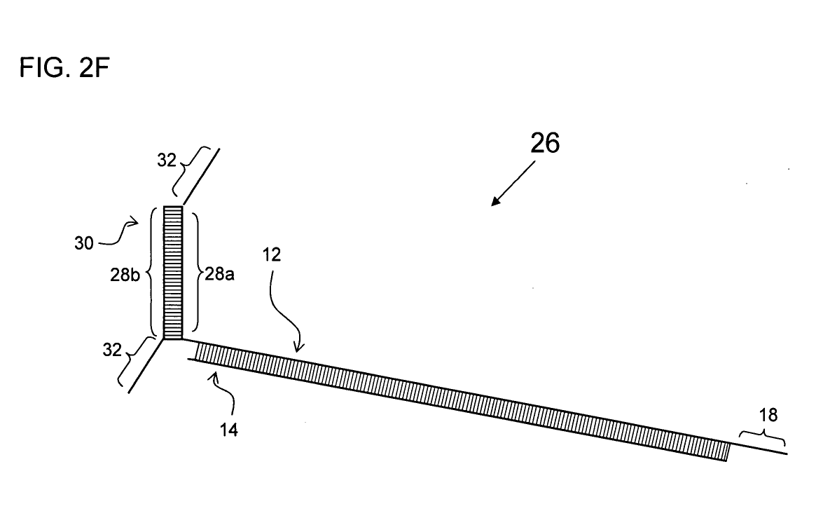

FIGS. 2A-2C show schematically three versions of an initiating monomer 26, each of which is shown with one binding arm 32. In FIGS. 2A and 2C, strand 30 has the one binding arm 32. In FIG. 2B, strand 12 has the one binding arm 32. FIGS. 2D-2F show schematically three versions of an initiating monomer 28 of the invention having two single-stranded hybridization binding arms. In FIG. 2D, strand 30 of initiating monomer 26 has both binding arms 32. In FIGS. 2E and 2F, strand 30 has one binding arm 32, and strand 12 has the other binding arm 32.

The initiating monomer is assembled by hybridizing the component polynucleotide strands, using methods well known to those skilled in the art. For instance, in one embodiment, the initiating monomer of the invention is formed by combining equimolar quantities of the three strands (e.g., 12, 14 and 30) in one solution, then heating and cooling the solution under controlled conditions that promote stringent hybridization. The hybridization product is optionally separated from non-hybridized molecules by standard techniques, such as gel filtration. Optionally, the double-stranded portions are crosslinked to further stabilize them, for example, by using psoralen and ultraviolet (UV) irradiation of the appropriate wavelength to promote psoralen-induced crosslinking of the double-stranded portions of the initiating monomer.

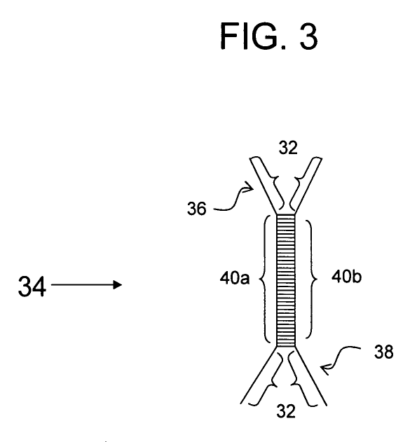

To assemble the polynucleotide dendrimer, one or more types of extending monomers, each possessing at least one binding arm and as many as four binding arms, are employed. An important aspect of the design of the extending monomers is that none of its single-stranded binding arms is complementary to the extension arm of the initiating monomer, which comprises the target-annealing site. In one embodiment, an extending monomer has four binding arms. As illustrated in FIG. 3, extending monomer 34 has two polynucleotide strands, strands 36 and 38. Each strand includes a trunk portion flanked by two binding arms. As shown in FIG. 3, strand 36 has trunk portion 40a, flanked by two binding arms 32. Strand 38 has trunk portion 40b, flanked by two binding arms 32. Trunk portion 40a is complementary in sequence to trunk portion 40b, which enables hybridization of strand 36 to strand 38 to form the extending monomer 34.

The extending monomers are prepared by hybridization of the two component polynucleotide strands, using procedures known to a skilled artisan. For example, approximately equimolar quantities of the two polynucleotide strands (e.g., 36 and 38) are combined in an aqueous solution, which is subsequently heated and cooled under controlled conditions to allow the hybridization of 40a to 40b and to maximize the yield of the extending monomer. The binding arms of the extending monomers are designed so that they will not hybridize with any other sequence in the component polynucleotide strands during the hybridization. Therefore, the binding arms in an extending monomer do not bind to each other. As described above for the initiating monomer, the extending monomer is optionally separated from non-hybridized component strands by standard techniques. Optionally, the double-stranded portions are crosslinked to further stabilize them, for example, by using psoralen and UV irradiation to promote psoralen-induced crosslinking of the double-stranded portions of the monomer.

The dendrimer of the invention is generally assembled by known methods in the art for constructing polynucleotide dendrimers. See, for instance, U.S. Pat. Nos. 5,175,270 and 6,274,723. The initiating monomer of the invention serves as the initial building block for assembling a polynucleotide dendrimer, and extending monomers are added to the initiating monomer to form the dendrimer in layers, where each is a layer of monomers. Consequently, the initiating monomer is at the approximate center of the polynucleotide dendrimer, particularly where the initiating monomer comprises three binding arms.

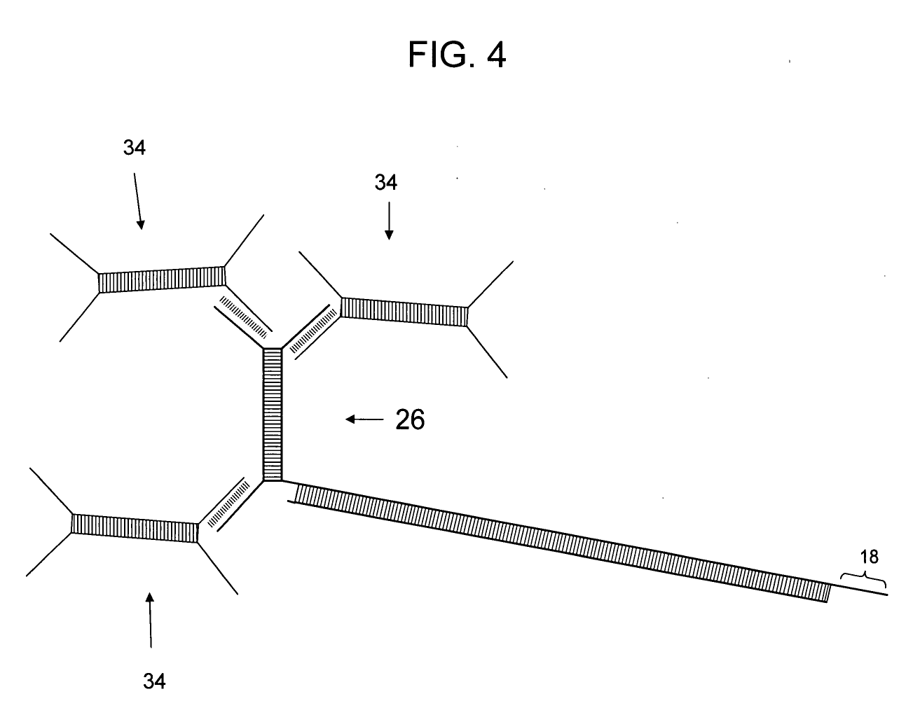

FIG. 4 depicts an initiating monomer of the invention with a first layer of three extending monomers hybridized to it, therefore providing a one-layer polynucleotide dendrimer, to which additional layers can be added. In the depiction, each extending monomer 34 of the first layer has one binding arm that is complementary to at least one binding arm of initiating monomer 26. The other binding arms of extending monomers 34 preferably do not hybridize to the extending monomers in the first layer. Therefore, during preparation of the first layer of a dendrimer, the extending monomers should hybridize only to the initiating monomer. Notably, the three extending monomers 34 in FIG. 4 are not identical to each other, because each layer of the polynucleotide dendrimer comprises at least two types of extending monomers. One type hybridizes to binding arms of the initiating monomer, or of the preceding layer, which are located at the 5′ end of a polynucleotide strand, and the other type hybridizes to binding arms located at the 3′ end of a strand. Upon binding to initiating monomer 26, each of the extending monomers 34 has three binding arms available for adding a second layer of extending monomers, and the extending monomers in the second layer are different from those in the first layer. Each layer of the dendrimer is thus added stepwise by the stepwise addition of layer-specific extending monomers. In certain embodiment, each extending monomer for a specific layer has only one of its binding arms that is complementary to single-stranded binding arms of the preceding layer. In certain embodiments, the extending monomers used to add a layer to the dendrimer can hybridize to the extending monomers of the preceding layer, but cannot hybridize to each other. In yet another embodiment, each extending monomer for a specific layer has only one of its binding arms that is complementary to single-stranded binding arms of the preceding layer, and the extending monomers in the specific layer cannot hybridize to each other.

In yet another embodiment, the extending monomers for adding a single layer to the dendrimer, or to the initiating monomer, consist of stoichiometric or super-stoichiometric quantities of at least two types of extending monomers. The types relate to a feature of single-stranded binding arms in the outer layer (i.e., the “acceptor layer”) to which the next layer of extending monomers is added to expand the dendrimer. Either the acceptor layer or the initiating monomer has two distinct types of binding arms: the type in which the polynucleotidal binding arms are oriented in the 5′-to-3′ direction with respect to their point of attachment to the dendrimer, and the type in which the binding arms are oriented in the 3′-to-5′ direction. Typically, the extending monomers for adding a single layer to the dendrimer, or to the initiating monomer, are designed to hybridize to either orientation. Therefore, one type of extending monomer is designed to hybridize specifically to one of the two orientations (for instance, 5′-to-3′) of binding arms, whereas another type of extending monomer is designed to hybridize specifically to the other orientation (3′-to-5′). Using a combination of layer-specific extending monomers of both types allows preparation of polynucleotide dendrimers of greater uniformity and denser branching, as described, for instance, in U.S. Pat. No. 5,175,270.

In an embodiment where every extending monomer has four binding arms, and one of the four binding arms hybridizes to the preceding layer of monomers, each layer of monomers can triple the number of binding arms available for hybridization of the next layer of extending monomers. When every layer is maximally saturated with extending monomers, the first layer will have 9 free binding arms, the second will have 27 arms, the third will have 81 arms, the fourth will have 243 arms, etc. A 2-layer polynucleotide dendrimer of the invention has up to 13 monomers (an initiating monomer, 3 monomers in the first layer, and 9 monomers in the second layer) and up to 27 free binding arms in the second layer. Consequently, a 3-layer dendrimer has up to 40 monomers and the third layer has up to 27 monomers.

The final layer of extending monomers forms the surface of the dendrimer, and some or all of the exterior layer of extending monomers have binding arms that are free to hybridize to another nucleic acid. In one embodiment, a 4-layer polynucleotide dendrimer is assembled using an initiating monomer of the invention, comprising a polynucleotidal extension arm and 3 binding arms, and various extending monomers that comprise 4 binding arms per monomer, totaling as many as 243 binding arms in the assembled dendrimer. The target-annealing site provided by the initiating monomer protrudes beyond the surface of the dendrimer. At least one of the extending monomers at the surface of the dendrimer is designed to hybridize to a complementary nucleic acid that is stably attached to a detectable label.

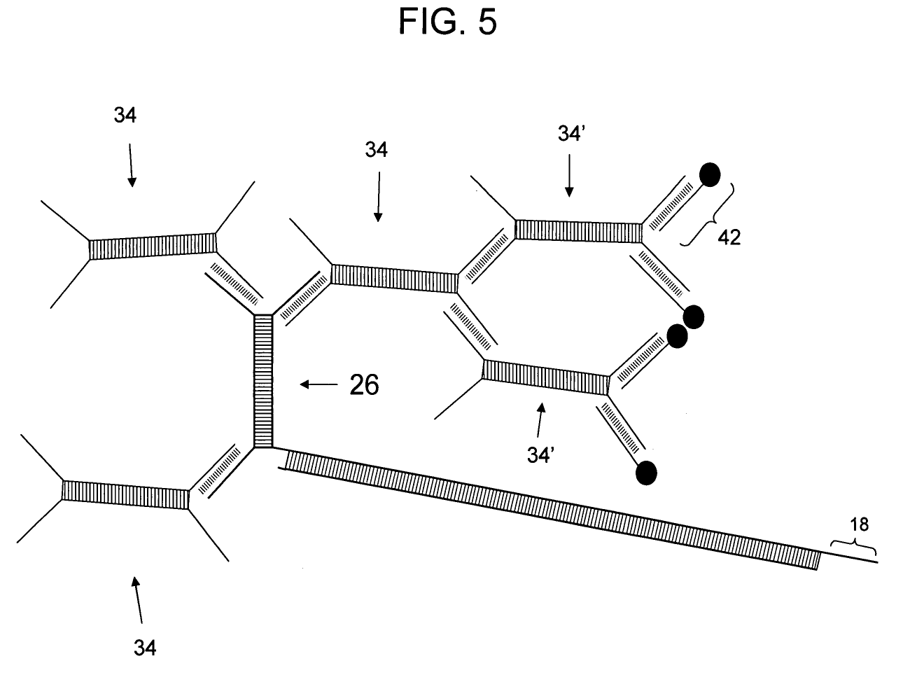

FIG. 5 illustrates a part of a 2-layer dendrimer, which is labeled by the hybridization of oligomer 42, which bears a detectable label (solid black circle) to free binding arms at the surface of the dendrimer. The extending monomers of the two layers are distinguished by being labeled 34 (for the first layer) and 34′ (for the second layer). The binding arm or arms at the surface, for binding of the detectable label, have a different nucleotide sequence than the target-annealing site of the extension arm of the initiating monomer in the dendrimer.

The dendrimers of the invention can be detected and quantified using any known method for labeling a polynucleotide, as well as methods not yet discovered. Any detectable label currently known to the skilled artisan, or those labels later discovered, can be used. In one embodiment, the detectable label is a photon-emitting compound. Non-limiting examples of a photon-emitting compound include phosphors, radioactive moieties and fluorescent moieties, such as rare earth chelates (e.g., europium chelates), Texas Red, rhodamine, fluorescein, FITC, fluo-3, 5 hexadecanoyl fluorescein, Cy2, fluorX, Cy3, Cy3.5, Cy5, Cy5.5, Cy7, dansyl, phycocrytherin, phycocyanin, spectrum orange, spectrum green, and/or derivatives of any one or more of the above. Other detectable labels include, but are not limited to, chromophores, chromogens, stable isotopes (for detection by mass spectroscopy or nuclear magnetic resonance spectroscopy), biotin, antigen epitopes, polynucleotidal aptamers, digoxigenin, among others.

After assembly, the polynucleotide dendrimer of the invention is optionally crosslinked to maintain and stabilize the structure of the dendrimer. Crosslinking hybridized regions between monomers (i.e., inter-monomer crosslinking) or between monomers and the nucleic acids that carry detectable labels, as well as between trunk portions (intra-monomer crosslinking), can stabilize the structure of the polynucleotide dendrimer. Such crosslinking chemistries are well known in the art. Non-limiting examples of suitable crosslinking agents include: psoralens (including but not limited to 8-methoxypsoralen and angelicin), mitomycin C, daunomycin, ethidium diazide, cisplatin, transplatin, carboplatin, 8-methoxypsoralen, mechlorethamine, oxaliplatin, and carbodiimide compounds, among others.

In one embodiment, the polynucleotide dendrimer is crosslinked using a psoralen type of crosslinking agent. Crosslinking using a psoralen or psoralens is conducted according to standard procedures. See, e.g., Cimino et al., Annu. Rev. Biochem. 54:1151-1193 (1985); Shi et al., Biochemistry 25:5895-5902 (1986); and Cimino et al., Biochemistry 25:3013-3020 (1986). See also U.S. Pat. No. 4,196,281.

The polynucleotide strands used in the monomers can be made using standard techniques for synthesis of nucleic acids. These techniques can be biological or chemical. The techniques and procedures are generally performed according to conventional methods in the art and in various general references (e.g., Sambrook et al., 2001, Molecular Cloning: A Laboratory Manual, Cold Spring Harbor Laboratory Press, Cold Spring Harbor, New York); Ausubel et al., eds., 1997, Current Protocols in Molecular Biology, John Wiley & Sons, New York, and Gerhardt et al., eds., 1994, Methods for General and Molecular Bacteriology, American Society for Microbiology, Washington, D.C.).

In one embodiment, polynucleotides are chemically synthesized. In another embodiment, polynucleotides are synthesized using the polymerase chain reaction (PCR). One PCR method suitable for generating single-stranded polynucleotides is multi-cycle PCR using a single primer, thereby amplifying a single strand.

The individual polynucleotide strands for preparing the initiating monomer and the extending monomers can have a wide variety of base compositions and lengths. In one embodiment, the base composition of the monomer is selected, such that its polynucleotide sequences, other than those intended to hybridize to each other (e.g., the monomer's trunk portions or extender portions) or to another sequence (e.g., the monomer's hybridization binding arms) within the dendrimer, have minimal sequence complementarity between each other and between the other components of the fully-assembled dendrimer, where the sequences are compared in all possible alignments. Further, the lengths of the individual strands are sufficiently long to provide the desired level of specificity for hybridizations between the specified sequences and to form stably hybridized duplexes, or hybridized duplexes sufficiently stable to be crosslinked, for addition stability, in which the duplexes are stably associated under the conditions in which the dendrimer is used. The strands are also sufficiently short to facilitate their preparation by chemical synthesis in vitro. The skilled artisan is familiar with polynucleotide sequence design and with considerations for avoiding undesired effects, such as intramolecular hybridizations or formation of other undesired secondary structures. See, also, U.S. Pat. No. 6,274,723 for sequence design for polynucleotide dendrimers. In one embodiment, the lengths of the component polynucleotide strands range from about 20 to about 200 nucleotides, with the exception of two strands of the initiating monomer, i.e., the strand comprising the extender portion and the target-annealing site, and the strand comprising the extender complement, which is complementary to the extender portion. The ranges on the extender portion and the target-annealing site have been discussed, supra.

In the initiating monomer of the invention, as well as in the extending monomers, each trunk portion can range from about 25 to about 500 contiguous base pairs. Preferably, the trunk portion ranges from about 30 to about 100 contiguous base pairs, and more typically, about 30 to about 50. In one embodiment, the trunk portions are composed of 35 base pairs. The single-stranded binding arms can be any length, ranging from about 12 to about 100 nucleotides, preferably about 20 to about 50 nucleotides and more preferably, about 25 to about 50 nucleotides. In one embodiment, the binding arms are 30 nucleotides long.

In one embodiment, target-annealing site 18a of polynucleotidal extension arm 10 comprises a sequence designed to bind directly to a particular target nucleic acid sequence. The dendrimer is, thus, specific to detecting that target nucleic acid sequence.

In another embodiment, target-annealing site 18a comprises a sequence designed to bind to target probe 20, shown schematically in FIG. 1B. As shown, target probe 20 has a nucleic acid sequence 18b complementary to target-annealing site 18a, and target-detecting portion 24. The use of a target probe is advantageous in that the dendrimer is not limited to detecting only a single target sequence. Rather, it can be used to detect virtually any target molecule, by modifying and appropriately designing the target probe.

In yet another embodiment, target-detecting portion 24 of target probe 20 is a nucleic acid sequence which is complementary to a target nucleic acid sequence. In one aspect, the target nucleic acid sequence is attached to a primer. The primer may be a sequence-specific primer or a collection of random primers. The primer is annealed to a complementary sequence in a nucleic acid, such as mRNA, in a sample, and the annealed primer may be used to amplify (i.e., by PCR) the sequence of all or part of the primer-bound nucleic acid. Thus, following the amplification, the amplified products each contain the target nucleic acid sequence, as installed by the primer. Alternatively, the annealed primer may be used for detecting a single copy of the primer-bound nucleic acid in the sample, where the annealed primer undergoes a primer extension procedure. In one variation, the amplified or primer-extended products are hybridized to a polynucleotide array. Subsequently, the array-bound products are detected by contacting the array with dendrimers of the invention which, in this embodiment, anneal specifically to the target nucleic acid sequences in the products. In another variation, the amplified or primer-extended products are first contacted with the dendrimers and subsequently are hybridized to an array. In another aspect, target-detecting portion 24 of target probe 20 is a primer sequence, in which case, target probe 20 is used either to amplify or to produce a single copy of a nucleic acid or variety of nucleic acids in a sample, in which each nucleic acid to be copied contains a sequence that is complementary to the primer sequence in target probe 20. After their production, the primer-mediated copies are detected by addition of the dendrimer.

In a related embodiment, target-detecting portion 24 of target probe 20 is a nucleic acid sequence which is an aptamer that binds to a protein or polypeptide target.

In still another embodiment, target-detecting portion 24 of target probe 20 is a polyamide. This embodiment is advantageous in detecting any molecule to which the polyamide binds. In one aspect, the polyamide is an antibody. In another aspect, the polyamide is a peptide aptamer.

In still another embodiment, there is provided a composition comprising two or more dendrimers, each of which targets a different target molecule and each of which is detectable labeled with different detectable labels, such that the dendrimers can be distinguished from each other. This composition is useful in any method where the simultaneous detection of two or more different target molecules in a sample is desired. Such multiplexing is particularly useful in microarray analysis, SNP detection, and cell flow cytometry, among others. In one embodiment, the two or more dendrimers, having distinguishably different detectable labels, are targeted to different molecules by the use of two target probes having different target-detecting portions.

The dendrimers are useful for nearly any method known for detecting a target molecule and/or quantifying the amount of a target molecule, for instance, a target nucleic acid or a target protein. In brief, a polynucleotide dendrimer is contacted with a sample comprising a target molecule. The polynucleotide dendrimer is allowed to bind to the target molecule under conditions that favor binding. The target molecule may be a nucleic acid, a protein or an antigen. In one embodiment, the target-annealing site of the polynucleotide dendrimer is designed to bind directly to a target molecule. In another embodiment, the target-annealing site is designed to bind to a complementary sequence in a target probe, and the target probe further comprises a target-detecting portion that binds to the target molecule. After binding the dendrimer to the target molecule, unbound polynucleotide dendrimer is typically removed, for instance, by washing. Optionally, the polynucleotide dendrimer is covalently linked to its target molecule after binding it. In some embodiments, the dendrimer comprises a detectable label prior to contacting the sample, while in others, the detectable label is attached to the dendrimer after the dendrimer has contacted the sample. In any case, the dendrimer is detected by any method suitable for distinguishing the detectable label. In some embodiments, the detectable label is a photo-detectable moiety, such as a fluorophore, a phosphor, a chromophore or a radioactive moiety. Optionally, the bound target molecule is also quantified. Quantification may be by a method independent of the dendrimer.

A non-limiting list of methods in which a polynucleotide dendrimer of the invention is useful includes: nucleic acid microarray analysis, gene expression profiles, sequencing, SNP detection, in situ hybridizations, such as FISH, Southern blots, Northern blots, ChIP assays, bead assays, biosensors, comet assays, dot blots, ELISA, protein arrays, immunohistochemistry, Western blots, tissue arrays, ELISA spots, cell flow cytometry and the assays disclosed in U.S. Pat. No. 5,487,973, among other. The dendrimers of the invention can be used with addressable beads, such as those sold be Luminex, which enable the separation of polynucleotides in a complex mixture based on sequence identity and hybridization affinity under stringent conditions. Advantageously, the dendrimer of the invention amplifies the detection signal of sparse polynucleotides hybridized to addresssable beads or other arrays in the art. In general, the dendrimers of the invention are useful in methods that involve quantifying a nucleic acid, protein or other target molecule, or that would otherwise benefit from the 1:1 stoichiometry of the inventive dendrimers.

The invention further provides kits useful in the practice of the methods of the invention. The kits comprise a polynucleotide dendrimer of the invention and an instructional material for the use or uses thereof. As used herein, an “instructional material” includes a publication, a recording, a diagram, or any other medium of expression which can be used to communicate the usefulness of the kit for using the polynucleotide dendrimer. The instructional material of the kit of the invention can, for example, be affixed to a container which contains the polynucleotide dendrimer or be shipped together with a container which contains the dendrimer. Alternatively, the instructional material can be shipped separately from the container with the intention that the instructional material and the polynucleotide dendrimer be used cooperatively by the recipient. In one embodiment, the kit further comprises an oligomer comprising a detectable label and a sequence complementary to specific binding arms of extending monomers in the kit or a separate kit. In another embodiment, the kit further comprises a target probe. In another kit embodiment, the target-detecting portion of the target probe is a nucleic acid sequence that is a primer. In another kit, the target-detecting portion of the target probe is a nucleic acid sequence that is complementary to a portion of an oligonucleotide comprising a primer, and the kit further comprises the oligonucleotide. Optionally, the kit further comprises a cloning vector comprising a sequence to which the primer can bind and, for instance, amplify the attached nucleic acid sequence.

The disclosures of each patent, patent application and publication cited or described in this document are hereby incorporated herein by reference, in their entirety.

While the foregoing specification has been described with regard to certain embodiments, and many details have been set forth for the purpose of illustration, it will be apparent to those skilled in the art without departing from the spirit and scope of the invention, that the invention can be subject to various modifications and additional embodiments, and that certain of the details described herein can be varied considerably without departing from the basic principles of the invention. Such modifications and additional embodiments are also intended to fall within the scope of the appended claims.

What is claimed is:

1. A polynucleotide dendrimer having only one target-annealing site, the dendrimer comprising: a polynucleotidal initiating monomer having a single-stranded target-annealing site projecting beyond the dendrimer's exterior layer and a plurality of polynucleotidal extending monomers, wherein the initiating monomer and the plurality of extending monomers are joined by hybridization.

2. The dendrimer of claim 1, wherein each extending monomer comprises initially a double-stranded trunk flanked by at least two hybridization binding arms that are single-stranded, such that when incorporated into the polynucleotide dendrimer, at least one of the hybridization binding arms of each extending monomer hybridizes to a complementary binding arm of the initiating monomer or another extending monomer.

3. The dendrimer of claim 1 comprising a detectable label.

4. The dendrimer of claim 3, wherein the detectable label is attached to an oligomer comprising a sequence which is complementary to a hybridization binding arm of an extending monomer of the exterior layer, and wherein the labeled oligomer is hybridized to the dendrimer.

5. The dendrimer of claim 3, wherein the detectable label is selected from the group consisting of: chromophore, chromogen, fluorophore, phosphor, radioactive moiety, biotin, antigen epitope, polynucleotide aptamer, isotope detectable by mass spectrometry, and isotope detectable by nuclear magnetic resonance spectroscopy.

6. The dendrimer of claim 5, wherein the detectable label comprises a photon-emitting label.

7. The dendrimer of claim 1, further comprising a target probe having a target-detecting portion and a sequence which is complementary to the target-annealing site, wherein the target probe is hybridized to the target-annealing site.

8. The dendrimer of claim 7, wherein the target-detecting portion comprises a nucleic acid sequence.

9. The dendrimer of claim 7, wherein the target-detecting portion comprises a polyamide.

10. The dendrimer of claim 9, wherein the polyamide comprises an antibody.

11. The dendrimer of claim 1, further comprising intermolecular crosslinks.

12. The dendrimer of claim 11, wherein the intermolecular crosslinks are psoralen mediated.

13. A polynucleotidal initiating monomer useful in preparing a dendrimer, the initiating monomer comprising:
a trunk;
at least one single-stranded hybridization binding arm covalently attached to the trunk; and
a polynucleotidal extension arm covalently attached to the trunk, wherein the extension arm comprises a double-stranded extender portion and a single-stranded target-annealing site.

14. A method of detecting a target molecule, the method comprising:
contacting the dendrimer of claim 1 with a sample containing a target molecule;
binding the dendrimer to the target molecule; and
detecting the dendrimer, thereby detecting the bound target molecule.

15. The method of claim 14, further comprising quantifying the bound target molecule.

16. The method of claim 14, wherein the target molecule comprises an antigen.

17. The method of claim 14, wherein the target molecule comprises a polynucleotide.

18. The method of claim 17, wherein the polynucleotide is within a polynucleotide array.

19. The method of claim 17, wherein the polynucleotide is attached to a solid bead or other solid support.

20. A kit comprising:
the dendrimer of claim 1; and
an instructional material.

21. The kit of claim 20, further comprising an oligomer comprising a sequence complementary to a hybridization binding arm of an extending monomer of the exterior layer of the dendrimer, and a detectable label.

22. The kit of claim 21, further comprising a target probe, wherein the target probe comprises a sequence that is complementary to the target-annealing site of the dendrimer and a target-detecting portion.

23. The kit of claim, 22, wherein the target-detecting portion comprises a nucleic acid sequence that is complementary to a portion of an oligonucleotide comprising a primer and wherein the kit further comprises the oligonucleotide.

24. The kit of claim 22, wherein the target-detecting portion is a nucleic acid sequence that is a primer.

25. A method of making a polynucleotide dendrimer having only one target-annealing site, the method comprising hybridizing a polynucleotidal initiating monomer and a plurality of extending monomers, wherein the polynucleotide initiating monomer comprises
a trunk;
at least one single-stranded hybridization binding arm covalently attached to the trunk; and
a polynucleotidal extension arm covalently attached to the trunk, wherein the extension arm comprises a double-stranded extender portion and a single-stranded target-annealing site, and
wherein each extending monomer comprises initially a double-stranded trunk flanked by at least two hybridization binding arms that are single-stranded, such that when incorporated into the polynucleotide dendrimer, at least one of the hybridization binding arms of each extending monomer hybridizes to a complementary binding arm of the initiating monomer or another extending monomer.

26. A composition useful for detecting two different target molecules in a sample simultaneously, the composition comprising a first polynucleotide dendrimer and a second polynucleotide dendrimer, wherein:
each polynucleotide dendrimer comprises a detectable label and a polynucleotidal initiating monomer having a single-stranded target-annealing site projecting beyond the dendrimer's exterior layer and a plurality of polynucleotidal extending monomers;
the initiating monomer and the plurality of extending monomers are joined by hybridization;
the single-stranded target-annealing site of the first polynucleotide dendrimer is different from the single-stranded target-annealing site of the second polynucleotide dendrimer; and
the detectable label of the first polynucleotide dendrimer is distinguishable from the detectable label of the second polynucleotide dendrimer.
